(19) United States
(12) Patent Application Publication
(10) Pub. No.: US 2011/0022041 A1
(43) Pub. Date: Jan. 27, 2011

(54) SYSTEMS AND METHODS FOR TITRATING RF ABLATION

(76) Inventors: Frank Ingle, Palo Alto, CA (US); Paul Roche, San Jose, CA (US)

Correspondence Address: Schwegman Lundberg & Woessner/ EPT-CRM;EPT-CRM, P.O. Box 2938, Minneapolis, MN, 55402, US

(21) Appl. No.: 12/835,367

(22) Filed: Jul. 13, 2010

Related U.S. Application Data
(60) Provisional application No. 61/228,295, filed on Jul. 24, 2009.

(51) Int. Cl. A61B 18/18

(52) U.S. Cl. 606/33

(57) ABSTRACT

An embodiment of a system for ablating tissue comprises an electrode configured for use to deliver RF power to ablate the tissue, and a heat flow sensor configured to provide a measurement of heat flow from the electrode to blood or irrigation fluid. According to some embodiments, the system further comprises an RF source configured to generate RF power connected to the electrode (P_{E}) to ablate tissue, and a controller configured to control a level of RF power and a duration for an ablation procedure. The controller is programmed to implement a process to estimate RF power dissipated in tissue (P_{T}), including calculating power loss due to convective heat flow (P_{CONV}) from the tissue through the electrode to the blood or the irrigation fluid to cool the electrode, and calculating the RF power dissipated in tissue (P_{T}) by subtracting P_{CONV} from P_{E}.

### CROSS REFERENCE TO RELATED APPLICATIONS

This application claims the benefit of U.S. Provisional Application No. 61/228,295, filed on Jul. 24, 2008, under 35 U.S.C. §119(e), which is hereby incorporated by reference in its entirety.

### TECHNICAL FIELD

This application relates generally to medical devices and, more particularly, to systems and methods related to radio frequency (RF) ablation systems.

### BACKGROUND

Aberrant conductive pathways disrupt the normal path of the heart's electrical impulses. For example, conduction blocks can cause the electrical impulse to degenerate into several circular wavelets that disrupt the normal activation of the atria or ventricles. The aberrant conductive pathways create abnormal, irregular, and sometimes life-threatening heart rhythms called arrhythmias. Ablation is one way of treating arrhythmias and restoring normal contraction. The sources of the aberrant pathways (called focal arrhythmia substrates) are located or mapped using mapping electrodes. After mapping, the physician may ablate the aberrant tissue. In radio frequency (RF) ablation, RF energy is directed from the ablation electrode through tissue to ablate the tissue and form a lesion.

Simple RF ablation catheters have a small tip and therefore most of the RF power is dissipated in the tissue. The advantage is that the lesion size is somewhat predictable from the RF power and time. However, the tissue can get very hot at the contact point, and thus there can be a problem of coagulum formation.

Various designs have been proposed to cool the ablation electrode and surrounding tissue to reduce the likelihood of a thrombus (blood clot), prevent or reduce impedance rise of tissue in contact with the electrode tip, and increase energy transfer to the tissue because of the lower tissue impedance. Catheters have been designed with a long tip for contact with blood to provide convective cooling through blood flow, which reduces the maximum temperature at the contact point. However, the amount of cooling depends on local blood velocity, which is uncontrolled and is generally not known. Since the convective heat transfer coefficient depends on the blood velocity, the tip temperature varies with blood velocity even at constant conduction power from tissue to tip. Thus, the electrophysiologist is less able to predict the lesion size and depth, as the amount of power delivered into the tissue is not known. Closed-irrigation catheters provide additional cooling to the tip, which keeps the tissue at the contact point cooler with less dependence on the local blood velocity. However, the added cooling further masks the amount of RF ablation power dissipated into the tissue. The tip temperature is poorly correlated to the tissue temperature. Open-irrigation catheters cover the tissue near the tip with a cloud of cool liquid to prevent coagulum in the entire region. However, more cooling fluid is used, which further masks the amount of RF power that enters the tissue.

If the amount of power entering the tissue is masked, then the size of the lesion cannot be accurately predicted. The RF power entering the tissue and the temperature profile versus time in the tissue is highly uncertain, which may contribute to under treatment or over treatment. If too much power is used, the tissue temperature may rise above 100° C. and result in a steam pop. Steam pops may tear tissue and expel the contents causing risk of embolic damage to the circulation. Additionally, the temperature differs throughout a volume of tissue to be ablated. A steam pop may occur in one part of the tissue volume before the tissue in other parts of the tissue volume reaches a temperature over 50° C. and is killed. As a consequence, power may be cautiously applied to avoid steam pop, and the tissue may be under treated resulting in the lesion being smaller than desired. The result of under treatment may be failure to isolate the tissue acutely or chronically, resulting in an inadequate clinical treatment of atrial fibrillation.

### SUMMARY

An embodiment of a system for ablating tissue comprises an electrode configured for use to deliver RF power to ablate the tissue, and a heat flow sensor configured to provide a measurement of heat flow from the electrode to blood or irrigation fluid. According to some embodiments, the system further comprises an RF source configured to generate RF power and connected to the electrode (P_{E}) to ablate tissue, and a controller configured to control a level of RF power and a duration for an ablation procedure. The controller is programmed to implement a process to estimate RF power dissipated in tissue (P_{T}). The process programmed in the controller includes calculating power loss from convective heat flow (P_{CONV}) from the tissue through the electrode to the blood or the irrigation fluid to cool the electrode, and calculating the RF power dissipated in tissue (P_{T}) by subtracting P_{CONV} from P_{E}.

According to a method embodiment, convective heat flow (P_{CONV}) is measured from the tissue through the electrode to the blood or the irrigation fluid to cool the electrode. RF power dissipated in tissue (P_{T}) is measured by subtracting P_{CONV} from generated RF power (P_{E}) for an ablation procedure. In some embodiments, a duration for applying RF power and a level of P_{E} for performing the ablation procedure is controlled using the calculated P_{T}. Thermal properties of tissue (e.g. at least one of a heat transfer coefficient or thermal diffusivity) are estimated, and the estimated thermal properties of tissue are used with the calculated P_{T} to control the duration and the level of P_{E} for performing the ablation procedure.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1A illustrates a chart of temperature against depth for a steady temperature in a static system; and FIG. 1B illustrates a graph of temperature against depth in a dynamic system.

FIG. 2 illustrates an example of heat transfer for an ablation electrode, according to various embodiments.

FIG. 3 illustrates a non-irrigation long ablation electrode with a gradient layer, according to various embodiments.

FIG. 4 illustrates an embodiment of a closed-irrigation electrode with a gradient layer with temperature measurement on each side of the layer, according to various embodiments.

FIG. 5 illustrates an open-irrigation electrode with a gradient layer with temperature measurement on each side of the layer, according to various embodiments.

FIG. 6 illustrates an electrode with thermocouples formed of different thermoelectric heat pump materials used to measure heat flow from the distal to proximal end of the electrode, according to various embodiments.

FIGS. 7-8 illustrate an embodiment of an ablation catheter which can be used with the tip perpendicular to the tissue or parallel to the tissue with the catheter deflected to the right or to the left.

FIGS. 9-11 illustrate various ablation catheter embodiments that use a single structure for the tip, with two temperature sensors T1 and T2 separated by some distance and aligned in the direction of the expected heat flow.

FIG. 12 illustrates a heat flow sensor with a gradient layer; and FIGS. 13A-B, 14A-B, 15A-C, and 16A-C illustrate various embodiments of the present subject matter that provide a heat flow sensor without a gradient layer.

FIG. 17 illustrates an embodiment of a mapping and ablation system, according to various embodiments of the present subject matter.

FIG. 18 illustrates a method for determining thermal properties of tissue, according to various embodiments.

FIG. 19 illustrates a method for determining thermal properties of tissue, according to various embodiments.

FIG. 20 illustrates a method for determining the time and power for RF ablation, according to various embodiments.

### DETAILED DESCRIPTION

The following detailed description of the present invention refers to subject matter in the accompanying drawings which show, by way of illustration, specific aspects and embodiments in which the present subject matter may be practiced. These embodiments are described in sufficient detail to enable those skilled in the art to practice the present subject matter. References to “an,” “one,” or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

During an RF ablation procedure, high RF current density near the electrode causes resistive heating. This heat is also transferred by conduction to surrounding tissue. Additionally, the electrode-tissue interface may be cooled by convection via blood flow or irrigation fluid. RF current is applied to tissue to locally heat a volume of the tissue to a temperature that kills cells (e.g. over 50° C. throughout the volume of tissue to be ablated). However, undesired steam pops occur if the temperature of a portion of the tissue rises to or above 100° C. Therefore, the temperature of the tissue to be ablated should be above 50° C. throughout the volume but should not reach 100° C. anywhere in the volume.

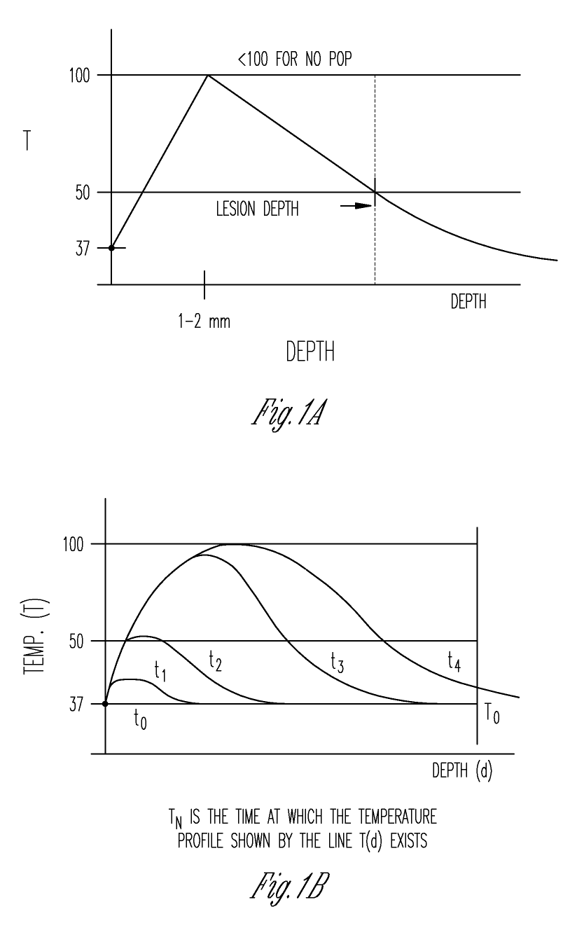

The temperature of tissue during the RF ablation procedure is not uniform. Most of the current density is concentrated at the tip of the electrode, and depends on electrode design. The RF current creates heating in the tissue in proportion to the square of the local current density, and heats the tissue. The part of the tissue closer to the surface tends to be convectively cooled by blood flow, and the deeper portions of the tissue have less current densities and thus experience less resistive heating. FIG. 1A illustrates a chart of temperature against depth for a steady temperature in a static system. The hottest temperature at steady state after application of the RF energy is approximately 1-2 mm deep. FIG. 1B illustrates a graph of temperature against depth in a dynamic system. This graph illustrates that as time progresses from t₀ to t₄, higher temperatures are observed at the same depths. Thus, the amount of time that RF power is applied is a factor in the depth of the lesion.

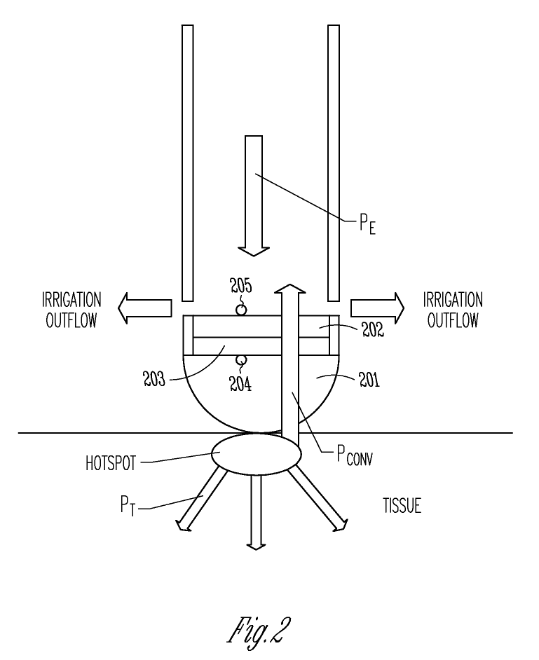

The present subject matter restores the ability of the electrophysiologist to predict the lesion without losing the advantages of irrigation to reduce clot formation. The amount of heat conducted through the tip away from the tissue being ablated is measured using a heat flow sensor. FIG. 2 illustrates an example of heat transfer for an ablation electrode, according to various embodiments. An open-irrigation system is illustrated, but the concepts described below apply to non-irrigated system and closed-irrigation systems, such as are discussed in this application. P_{T} is the power into the tissue, P_{E} is the RF power delivered to the catheter tip electrode, and P_{CONV} is the power conducted from the distal tip toward the proximal portion for dissipated by convection into irrigation fluid or blood flow. The power delivered to the tip (P_{E}) is either dissipated in tissue (P_{T}) or dissipated in blood or irrigation fluid (P_{CONV}) (e.g. P_{T}=P_{E}−P_{CONV}). FIG. 2 illustrates an RF electrode tip 201, a heat sink 202, and a gradient layer 203 positioned between the RF electrode tip 201 and heat sink 202. RF power is delivered from the RF electrode tip 201 to the tissue. A hot spot forms in the tissue near the tip. Some of the heat is conducted further into the tissue, as represented by P_{T}, and some of the heat is conducted to the electrode tip 201, and then through the gradient layer 203 and to the heat sink 202 for dissipation to irrigation fluid or blood. The illustrated system also includes a first thermocouple 204 and a second thermocouple 205 positioned on opposite sides of the gradient layer 203. The electrode tip 201 and heat sink 202 are fabricated using materials that are very good conductors of heat. Thus, the temperature sensed using the first thermocouple generally represents the temperature at the tissue/electrode interface. The heat transfer properties of the gradient layer 203 are known, such that the energy P_{CONV} traveling from the tip 201 to the heat sink 202 can be calculated using the temperature sensed by the thermocouples 204 and 205 on each side of the gradient layer 203. This energy P_{CONV} dissipates into the irrigation fluid or blood. Using this information and the known power generated by the RF generator, the amount of power which flows into the tissue P_{T} can be estimated relatively independent of the blood velocity and irrigation fluid. Thus, physicians will be better able to control the formation of the lesion with the estimate of P_{T}. Various embodiments are illustrated below.

The heat which flows through the tip is carried away by convection to the blood or irrigation fluid. The temperature gradient across the gradient layer is ΔT=(T2−T1), and the heat flow through the gradient layer is P_{CONV}=k1*ΔT, where k1 is a constant related to the Seebeck coefficient of the thermocouple, the area of the gradient layer, and the thickness of the gradient layer. Thus, the heat dissipated by convection can be measured independent of the blood velocity or irrigation fluid flow that cools the electrode.

The RF power is measured or controlled by the RF generator as P_{E}, and by subtracting the power convected away leaves the power dissipated in the tissue: P_{T}=P_{E}−P_{CONV}. With this simple correction to the RF power, the electrophysiologist knows an accurate estimate of the amount of RF current dissipated in the tissue independent of the blood velocity and irrigation fluid, and yet the tip is still cooled by the convection (blood and/or irrigation fluid). This makes the size and depth of lesions more predictable, while still affording the protection of a cooled tip. In addition, knowing the tip temperature and the power flowing into the tissue P_{T} allows the temperature versus depth in the tissue to be estimated as it changes with time.

The RF current heats tissue in a way which depends on tissue conductivity and tip geometry, and may be estimated. Passive heat transfer also occurs within the tissue as a function of tissue properties, which may be estimated. Knowing the temperature profile versus time allows us to estimate the depth at which the tissue is killed, as evidenced by the tissue temperature being 50° C. or above. The calculated temperature versus depth profile can also be monitored to avoid overheating of the tissue which can cause a steam pop. This can occur when the tissue temperature anywhere in the tissue exceeds about 100° C.

These estimates can be used to assist the electrophysiologist in choosing the time and power for the procedure for a desired lesion depth. The calculation can be augmented with animal experiments to bring the parameters closer to the actual values for living tissue.

For any desired lesion depth, there is a minimum power needed to achieve the lesion at infinite time, and a maximum power at which the lesion can be achieved without a steam pop occurring. A quicker treatment uses more power within these limits. Some embodiments choose a power within the range that produces a lesion depth which minimizes the time required and minimizes the likelihood of a steam pop. Some embodiments use a power between these limits for each desired lesion depth, which will then determine the treatment time needed to reach the desired depth without a steam pop.

For a desired lesion depth, there is a maximum power which will allow the temperature to rise to over 50° C. at that depth without causing the temperature to exceed 100° C. anywhere in the tissue. For deeper desired lesions, the power is lowered to avoid overheating anywhere in the tissue from the tissue/electrode interface to the depth of the lesion. There is also a specific time required for the temperature at the desired depth to reach 50° C. The deeper the desired lesion, the longer it will take to form the lesion. A table can be created with estimated tissue properties to guide the electrophysiologist in making lesions of a desired depth. The table would identify the power and time for applying ablation energy to achieve a lesion of a desired depth in tissue with estimated thermal properties. The accuracy of this information depends on the extent and accuracy of the tissue thermal properties of the tissue accurately. There is tissue variation, which affects the accuracy of information in the table.

The accuracy of the table can be improved to some degree by using the measured data from the actual patient to make corrections to the parameters. When a constant level of RF power is first applied, the temperature of the tip will increase slowly as the tissue is heated. The thermal properties of the tissue can be estimated before power ablation by first applying a constant level of RF power to slowly increase the temperature of the tip as the tissue is heated. The thermal properties of the tissue are estimated using the initial rate of temperature rise, the final temperature reached, and the constant power applied. The parameters of the ablation (e.g. power and/or time) can be adjusted using this estimate of the thermal properties for the tissue.

If a small amount of power is applied as a step function, the tissue will heat to a constant temperature. The time until the temperature stabilizes can be used to calculate the heat transfer coefficient k of the tissue. If power is applied as a step function and the initial rate of rise of the transient increase in temperature at the tip is measured, the thermal diffusivity α of the tissue can be calculated. The thermal conductance κ and the thermal diffusivity α are related by the following equation: α=κ/(ρCp), where alpha α is the thermal diffusivity, and κ is the heat transfer coefficient. The heat capacity of the tissue ρCp depends on the density ρ and specific heat capacity Cp of the tissue.

The RF ablation process heats tissue at the electrode tip. Heat flows from the electrode tip/tissue interface through the gradient layer and into the heat sink, where it is dissipated into the blood and/or irrigation fluid. The material for the gradient layer is typically a much poorer conductor than the metal of the tip and the shaft. By measuring the temperature difference across the gradient layer and knowing the dimensions and heat conductivity of the gradient layer, the heat flow through the gradient layer can be calculated. This heat flow through the gradient layer represents the heat lost from the ablated tissue, which has been heated by the RF power, where the lost heat flows through the shaft for dissipation through convection into the blood. Since the electrical RF power delivered by the RF generator is known and the heat lost by convection can be measured, the remaining RF heat which is dissipated in the tissue can be calculated: P_{T}=P_{E}−P_{CONV}, where P_{T} is the power in the tissue, P_{E} is the RF power, and P_{CONV} is the heat carried away by the fluid. Thus, the electrophysiologist can know how much power is delivered to the tissue despite the convection cooling to the blood and/or irrigation fluid, which would otherwise blind the electrophysiologist to the RF power being delivered into the tissue. With this information, the electrophysioligist is better able to estimate the size and depth of the lesion.

Any heat which conducts from tissue to tip is measured by the gradient layer calorimeter or other heat flow sensor. The heat flow from tissue to tip can be measured and subtracted from the measured RF electrical power to obtain the amount of heat dissipated in the tissue. Additionally, thermal properties of the tissue can be estimated using the tip temperature. This information can be used to predict the lesion size and depth. If the tissue properties were known, the power could be measured and used to calculate the temperature profile versus depth at any time. Unfortunately, tissue varies. However, adjustments can be made by measuring the tip temperature. A simulation will identify what the tip temperature should be based on the assumed thermal properties for the tissue. An error in tip temperature can be used to correct assumptions about the tissue thermal properties. Thus, we can make a better estimation of temperature profile versus depth at any time. With a more accurate estimate of tissue temperature versus depth and time, the lesion depth versus time can be estimated more accurately.

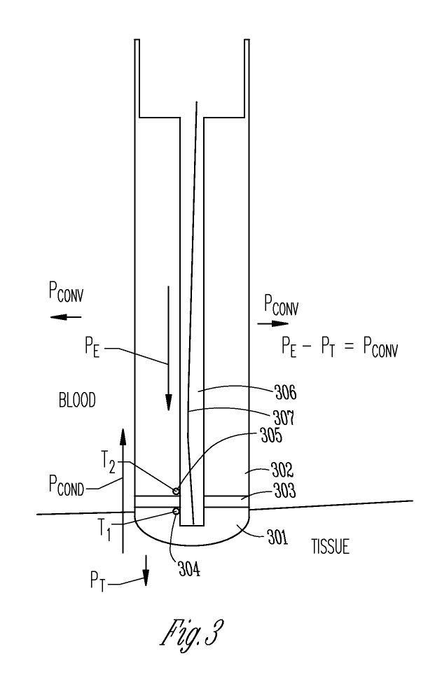

FIG. 3 illustrates a non-irrigation long ablation electrode with a gradient layer, according to various embodiments. The RF electrode tip 301 is constructed using a metal with high thermal conductivity and high electrical conductivity. For example, some embodiments use platinum plated copper for an RF electrode. A gradient layer 303 is provided by a thin layer of material whose thermal conductivity is low compared to the metal tip. The gradient layer 303 is attached to the metal tip on its proximal face. The material properties (κ) and thickness (t) of the gradient layer depend on the desired temperature change ΔT across the gradient layer for the maximum amount of heat flow (P): P=(κAΔT)/t, where A is area of disk. In some embodiments, the desired temperature change ΔT is 5° C.-10° C. (a temperature change that can be accurately detected and processed using current technology). Another metal cylinder 302 is on the proximal side of the gradient layer. The metal cylinder functions as a heat sink and transfers the conducted heat to the flowing blood by convective heat transfer. There are temperature sensors 304 and 305 (e.g. thermocouples) on each side of the gradient layer 303. A central hole 306 down through the metal cylinder and gradient layer allow an electrical connection 307 for the RF ablation current between an RF generator and the RF electrode tip 301.

The thermocouples 304 and 305 are used to measure the temperature in two distinct locations on the electrode. The distal thermocouple 304 measures the temperature of the electrode near the tissue interface, and thus provides a measurement of tissue temperature. The proximal thermocouple 305 measures the temperature of the electrode at a more proximal end of the electrode. The thermocouples 304 and 305 can be used to determine heat flow from the distal portion of the electrode near the tissue interface toward the proximal portion of the electrode.

FIG. 4 illustrates an embodiment of a closed-irrigation electrode with a gradient layer with temperature measurement on each side of the layer, according to various embodiments. The RF electrode tip 401 is constructed using a metal with high thermal conductivity and high electrical conductivity. For example, some embodiments use platinum plated copper for an RF electrode. A gradient layer 403 is provided by a thin layer of material whose thermal conductivity is low compared to the metal tip. The gradient layer 403 is attached to the metal tip on its proximal face. The material properties (κ) and thickness (t) of the gradient layer depend on the desired temperature change ΔT across the gradient layer for the maximum amount of heat flow (P): P=(κAΔT)/t, where A is area of disk. In some embodiments, the desired temperature change ΔT is 5° C.-10° C. (a temperature change that can be accurately detected and processed using current technology). Another metal cylinder 402 is on the proximal side of the gradient layer. The metal cylinder functions as a heat sink and transfers the conducted heat to the flowing blood by conductive heat transfer. There is also a temperature sensor 404 and 405 on each side of the gradient layer 403. A central hole 406 down through metal cylinder and gradient layer allow an electrical connection 407 for the RF ablation current between an RF generator and the RF electrode tip 401. The metal cylinder also includes fluid passages through which cooling fluid is delivered toward the distal end of the electrode and returned. In the illustrated embodiment, heat is conducted from the electrode tip 401 through the gradient layer to fluid in region 408 as well as to heat sink 402. Cooling or irrigation fluid is pumped from a reservoir, not illustrated in FIG. 4, through passage 409 to region 408 and returned to the reservoir through 410. The temperature of the fluid in the reservoir is controlled. Thus, the fluid transfers the heat from the electrode toward the temperature-controlled reservoir.

The thermocouples 404 and 405 are used to measure the temperature in two distinct locations on the electrode. The distal thermocouple 404 measures the temperature of the electrode near the tissue interface, and thus provides a measurement of tissue temperature. The proximal electrode 405 measures the temperature of the electrode at a more proximal end of the electrode. The thermocouples 404 and 405 can be used to determine heat flow from the distal portion of the electrode near the tissue interface toward the proximal portion of the electrode.

As heat flows up from the tissue, it is carried away by the flowing liquid (e.g. saline). The temperature difference across the gradient layer is used to calculate the amount of heat P_{CONV} which flows from tip to the irrigation fluid. A gradient layer is interposed between the tip and the flowing closed-irrigation fluid. The heat flow from the tip can be calculated from the temperature gradient across the layer and the size and thermal properties of the layer.

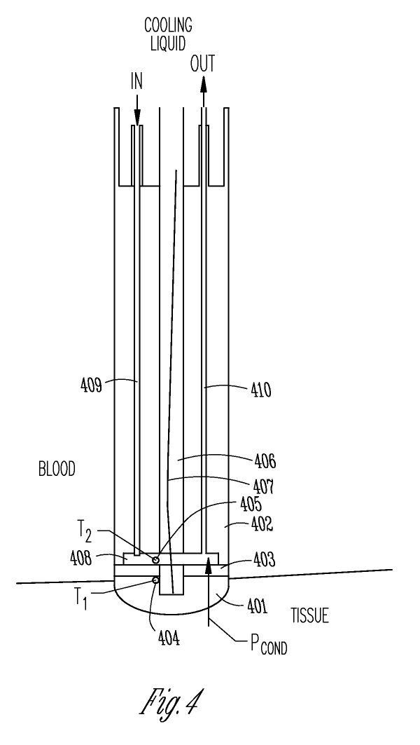

FIG. 5 illustrates an open-irrigation electrode with a gradient layer with temperature measurement on each side of the layer, according to various embodiments. The irrigation fluid is allowed to cool the tip and then flows out of the catheter near the distal end. The illustrated embodiment includes an RF electrode tip 501 connected to an RF generator via a conductor 507, a gradient layer 503, and a good conductor of heat functioning as a heat sink 511 on the proximal side of the gradient layer. The ablation catheter includes a gradient layer 503 between the metal RF ablation tip 501 and the distal shaft 512 of the catheter. Any heat which conducts from tissue to tip is measured by the gradient layer calorimeter using the temperature sensors 504 and 505, and then heats the liquid that flows through the distal shaft 512 and then flows out of the irrigation holes 513. The heat flow from tissue to tip can be measured and subtracted from the measured RF electrical power to obtain the amount of heat dissipated in the tissue. Additionally, thermal properties of the tissue can be estimated using the tip temperature. This information can be used to predict the lesion size and depth.

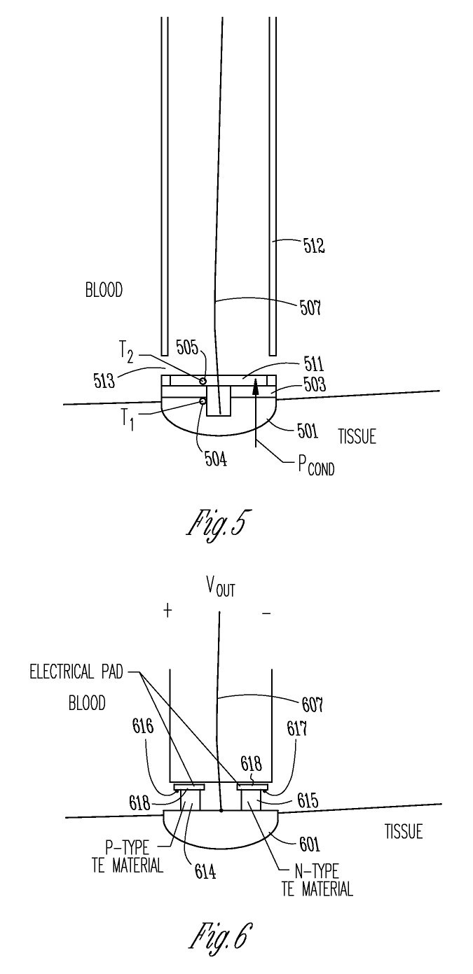

FIG. 6 illustrates an electrode with thermocouples formed of different thermoelectric heat pump materials used to measure heat flow from the distal to proximal end of the electrode, according to various embodiments. Small blocks of material 614 and 615 are used to form a thermocouple. According to various embodiments, the material is the same as used in commercial thermoelectric heat pumps: doped bismuth telluride and doped antimony selenide. The same properties which make the material efficient as a heat pump make it more sensitive as a heat flow measuring device. The two blocks can be visualized as two wires of a thermocouple. The Seebeck coefficient is about 100 uV/degree C., and the structure is easily capable of conducting a large heat flow with small temperature gradient.

In the illustrated embodiment, the tip 601 is a copper tip connected to an RF generator via a conductor 607. The two blocks 614 and 615 are attached to the copper tip 601, which also acts as an electrical conductor to connect the distal ends of the two blocks in series electrically. Thus the connection of the two bottom sides of the blocks 614 and 615 via tip 601 forms one leg of a thermocouple. The top sides of the two blocks are connected to two wires 616 and 617 which are used to sense the voltage generated by the thermocouple as heat flows through it generating a temperature difference. The output voltage is proportional to the heat flow and the material properties of the block material. The temperature gradient achieved depends on the thermal conductivity of the blocks and their dimensions.

A plate 618 is affixed to the top of the blocks to prevent the irrigation water from flooding the blocks and corroding the materials. This plate is made of a material which is a good thermal conductor and a poor electrical conductor, such as alumina (Al₂O₃). This type of heat flow measuring sensor could be used in a non-irrigated ablation catheter, in a closed-irrigation catheter, and in an open-irrigation catheter.

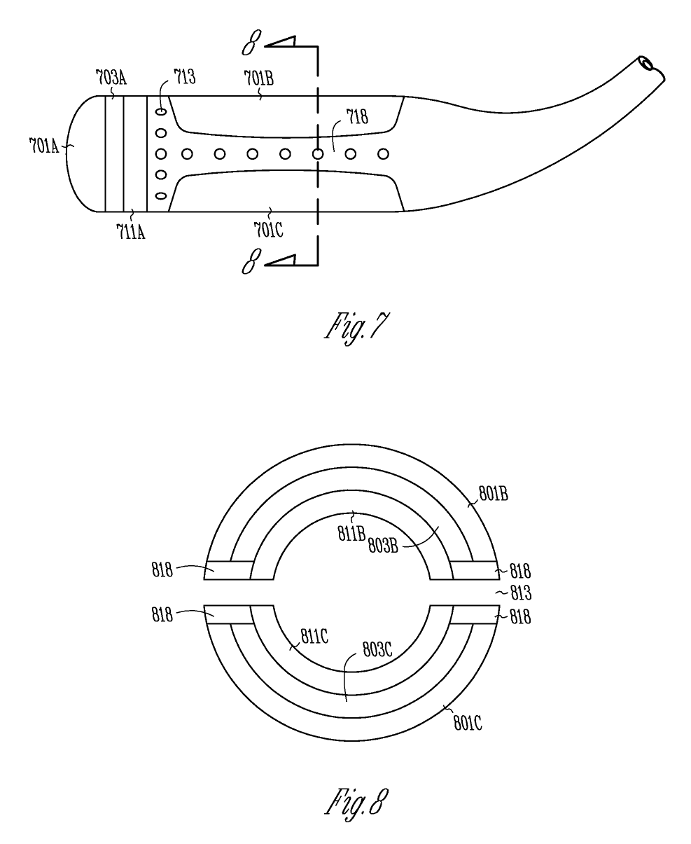

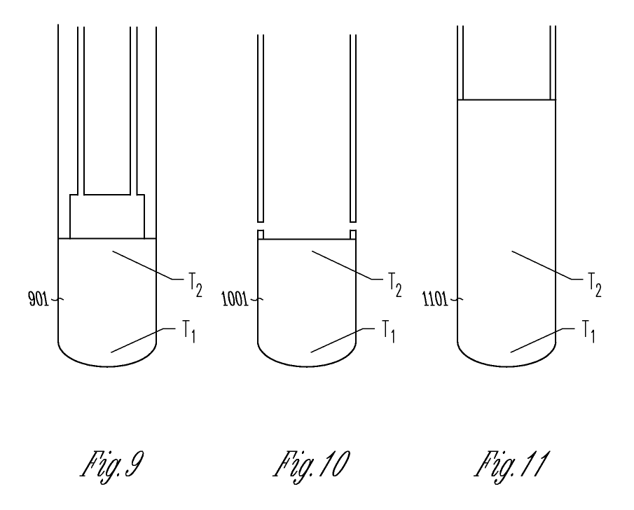

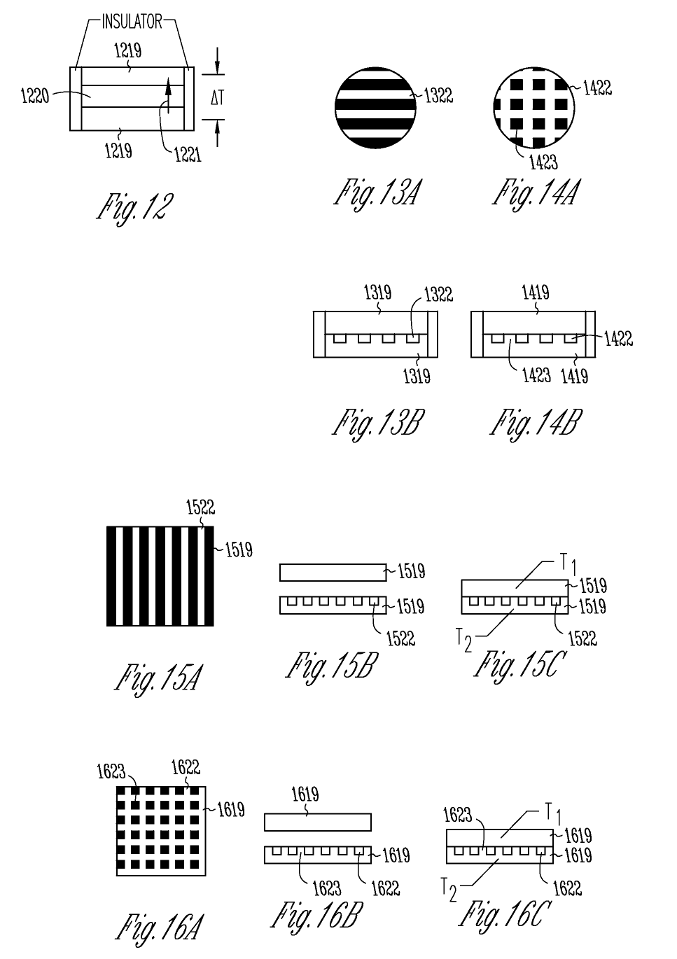

FIGS. 7-8 illustrate an embodiment of an ablation catheter which can be used with the tip perpendicular to the tissue or parallel to the tissue with the catheter deflected to the right or to the left. Two temperature sensors are included in the tip, two on the right side, and two on the left side. With reference to both FIGS. 7 and 8, three gradient layer structures facilitate measurement of the heat flowing into the catheter from the tip using electrode 701A, gradient layer 703A and heat sink 711A, from the right side using electrode 701B or 801B, gradient layer 803B and heat sink 811B, or from the left side using electrode 701C or 801C, gradient layer 803C and heat sink 811C. These measurements allow the user to know how much of the heat (from RF ohmic heating of the tissue) is transferred from the tissue via convection and how much of the heat is contributing to lesion formation. This measurement is expected to be relatively independent of the velocity of blood nearby, and thus the lesion depth and size can be estimated much more accurately. The heat which flows from the tissue through the gradient layer is measured, independent of how well the heat is convected away by the blood flow. There may be a small error if the blood cools the side margins of the side electrodes, but this can be reduced by reducing the side electrode size so that most of the electrode surface faces the tissue. The multi-electrode concept, with gradient heat flow measurement, may be used with open-irrigated catheters, closed-irrigated catheters, or non-irrigated catheters.

The illustrated catheter may be used end fired via electrode 701A when it is held perpendicular to the tissue, or side fired via electrode 701B or 701C, when it is pressed parallel to the tissue. Since the tip may be deflected right or left, it may fire to the right side or to the left side. The gradient layer method described earlier may be extended to cover this type of catheter, whether irrigated (open or closed) or non-irrigated.

The sides of the gradient layer are electrically and thermally insulated from the blood, so that all the power conducted upwards into the tip flows through this gradient layer. The temperature difference between the tip and the upper layer is the temperature gradient, and the power flowing from tip up to the upper layer is a function of the temperature gradient, the geometry, and the material's thermal conductivity of the gradient layer. Thus, the power which flows from the tip to the upper layer is proportional to the temperature difference across the gradient layer. All the heat which flows from the tip to the upper layer is carried away via the convective cooling of the blood flow or the irrigation fluid.

In the case of an open-irrigated catheter, there will be a multiplicity of small holes 713 around the periphery of the catheter tip, just above the level of the top conductive layer. Thus, any heat which flows up from the tissue will be conducted into the open-irrigation fluid and then out to the region just above the tissue around the tip, which will serve to cool it and also dilute the blood with heparinized saline. The result is to lower the likelihood that coagulum will be formed on the surface of the tissue. Thus, the temperature gradient can be measured and the power flowing upwards from the tip can be calculated using a predetermined calibration constant. Using this information, the power flowing into the tissue can be calculated by taking the RF power measured by the RF generator and subtracting from it the gradient layer calculated power, leaving the power actually deposited into the tissue. This assumes that the metal tip is not in contact with the blood. It is sized and shaped so that most of the electrode is in direct contact with the blood, and no electrical current or heat flows from the electrode directly into the blood.

The generator may activate anyone of the three electrodes: 701A, 701B or 701C. The electronics can sense which electrode is in contact with the tissue by applying a small current, calculating the impedance and RF power being delivered, and the tip temperature. In this fashion, the catheter acts as a hot film anemometer, and its temperature is inversely proportional to the heat transfer coefficient in the medium touching the electrode. In addition, this allows power to be driven through only the side of the electrode which faces the tissue. Since the impedance of the blood is much lower than that of tissue, more than half of the RF power usually flows into the blood for no purpose, and may create coagulum at the electrode. Choosing to drive RF only into the tip or only the side in contact with the tissue will reduce possible problems with coagulum in addition to measuring the actual power into the tissue.

In an embodiment, the sensor system can determine which electrode touches the tissue and apply RF power to that location. In some embodiments, a sensor in the catheter handle is used to determine which direction the tip is deflected, and makes connection to the proper RF electrode.

The catheter tip may be deflected right or left, so there is an ablation electrode shown on the top side and the underside. It is also possible to provide a side electrode only on a single side, and rotate the catheter to bring the correct side of the catheter in contact with the tissue, so the electrode is pressed into the tissue. The tip is shown on the left, and it has the metal tip 701A, gradient layer 703A, and metal conductive layer 711A. In the center of the picture is an electrode 701C on the lower edge in contact with the tissue. With reference to both FIGS. 7 and 8, an insulating region 718 or 818 is shown so that the gradient layer heat flow sensors can operate independently, and small holes 713 or 813 are shown in a row on the side of the catheter, to allow flow of the open-irrigated catheter fluid. There is a similar row on the opposite side. The position of these holes also allows the fluid to cool the tissue next to the catheter and to keep the blood in contact with the tissue diluted with heparinized fluid, to further prevent the formation of clot or coagulum.

FIG. 8 shows a cross section of the catheter at section 8-8 of FIG. 7. The lower electrode is a highly thermally conductive material such as platinum, copper, silver or aluminum, and may be plated with another metal such as platinum or gold. It also serves as the RF electrode when the catheter is used as a side fired device. A temperature sensor is located in this layer. Gradient layer 803C is a much poorer thermal conductor than the outer electrode 801C. Closer to the center of the catheter is the isothermal metal half cylindrical shell 811C. A temperature sensor is located in this layer. When RF current is driven from electrode 801C into the tissue, heat is generated in the tissue near the electrode surface, and then flows passively as determined by the temperature gradient. The heat flowing from the tissue to the catheter flows through the electrode 801C, then through the gradient layer 803C and into the isothermal metal half cylindrical shell 811C. The temperature gradient across the gradient layer is proportional to the thermal conductivity and geometry of the gradient layer. Thus, with a suitable calibration constant, the power which flows from the tissue into the catheter may be measured. The heat that flows from the tissue into the catheter is dissipated by the irrigation fluid in an open or closed-irrigated catheter, or into the blood if no irrigation is provided. RF electrode 801B, gradient layer 803B and isothermal metal half cylindrical shell 811B operate in a similar manner.

Thus, this catheter provides three simultaneous measurements: heat flow from the tip into catheter, heat flow from the left side electrode into the catheter, and heat flow from the right side electrode into the catheter. In addition, the catheter measures the electrode temperatures at the tip, the left electrode, and the right electrode.

FIGS. 9-11 illustrate various ablation catheter embodiments that use a single structure for the tip, with two temperature sensors T1 and T2 separated by some distance, and aligned in the direction of the expected heat flow. FIG. 9 illustrates a closed-irrigation system, FIG. 10 illustrates an open-irrigation system, and FIG. 11 illustrates a non-irrigation system. Since all materials have an imperfect thermal conductivity, heat flowing in the material creates a temperature gradient, with the heat flowing in the direction from the warmer of the two sensors toward the cooler of the two. This is a simpler structure than embodiments that incorporate a distinct gradient layer, and the calibration constant of the device will be less predictable. However, these embodiments can be calibrated and the calibration constant depends on the conductivity of the material and the geometry, both of which can be controlled. The response will be quicker if the temperature sensors are closer together and closer to the distal end of the RF ablation tip. The two sensors can be individual sensors such as thermocouples or thermistors. They can also be combined into a single assembly which is inserted into the tip axially into a hole and then attached to achieve good thermal conductivity with the wall. The illustrated tip is a metal with high thermal conductivity, such as copper, silver, or aluminum, and may be coated with another metal such as platinum for good performance in measuring electrograms between applications of RF ablation.

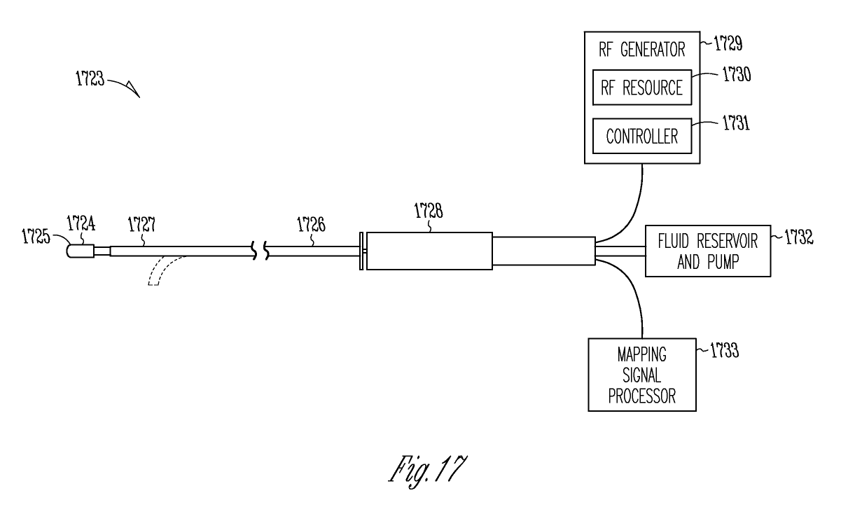

A gradient layer heat flow sensor can be used to measure the heat which flows from the tip to the cooling mechanism of an RF ablation catheter by use of a gradient layer heat flow sensor. With reference to FIG. 12, a typical gradient layer heat flow sensor is a sandwich consisting of a good thermal conductor 1219 on each side and a much poorer heat conductor 1220 in the middle. As heat flows, as illustrated by arrow 1221, through the sandwich from face to face, it flows through the gradient layer. The temperature difference (Delta T) measured across this gradient layer is then proportional to the heat flow from face to face of the sandwich. A temperature sensor is provided in thermal contact with the upper layer and the lower layer. The thermal gradient within the good thermal conductors is small and most of the thermal gradient occurs within the gradient layer which is identified. The three layers should be relatively thin compared to the diameter, and relatively thin in absolute terms since the time response of the sensor is highly dependent on the thermal delay caused by heat diffusion through the layers. It can be difficult to obtain a material for the gradient layer with a thermal conductivity in the right range of values, and which can be reliably bonded to the outer layers.

With reference to FIGS. 13A-B, 14A-B, 15A-C, and 16A-C, various embodiments of the present subject matter provide a heat flow sensor without a gradient layer. It is noted that these figures do not necessarily illustrate the grooves drawn to scale. At least one of the thermal conductors has parallel grooves 1322 milled into its face. The two layers 1319 are then oriented face to face with the grooves perpendicular to each other. The two layers 1319 are then bonded together, by methods such as being plated with solder and flux and then heated above the melting point of the solder. The gradient layer is thus formed by the grooved area of the layer. If the face area of the grooved layer is 90% open and the grooves have approximately perpendicular walls, then the thermal conductivity of the gradient layer thus formed will be only 10% of the bulk material.

With reference to FIGS. 14A-B and 16A-C, if the grooved side is then milled again perpendicular to the original grooves to provide a cross pattern of grooves 1422, 1622, then the resulting field of small posts 1423, 1623 will have a thermal conductivity of 1% of the bulk material. In addition, the two layers 1419, 1619 are of the same material so a bond is easy to make and there is no thermal stress in the layer as the temperature changes.

For example, it is desirable to have a very high thermal conductivity for the outer layers, consistent with using copper, silver for the layer. The layer may be coated with another metal to provide corrosion resistance, such as gold plating or platinum plating. TABLE 1 lists thermal conductivities for good conductors which also might be considered for use in the body, and also lists thermal conductivities for water, blood, muscle and fat.

TABLE 1

| | Thermal Conductivity |
|---|---|
| Material | Watts/(cm * degree C.) |
| Silver | 4.28 |
| Copper | 4.01 |
| Aluminum | 2.36 |
| Magnesium | 1.57 |
| Silicon | 1.3 |
| Brass | 1.01 |
| Iron | 0.83 |
| Platinum | 0.73 |
| Gold | 0.61 |
| Tantalum | 0.57 |
| Water | 6.28E−03 |
| Blood | 5.70E−03 |
| Muscle | 4.80E−03 |
| Fat | 3.70E−03 |

The thickness of the gradient layer cannot be too thick or there will be too much temperature drop across the gradient layer. The maximum thickness of the tip without a gradient layer for a temperature drop of 5° C. with a power across the gradient of 20 watts are illustrated in TABLE 2.

TABLE 2

| | Tip Thickness in mils |
|---|---|
| Material | Δ5 = 5° C., P = 20 w |
| Silver | 30 |
| Copper | 28 |
| Aluminum | 17 |
| Magnesium | 11 |
| Silicon | 9 |
| Brass | 7 |
| Iron | 6 |
| Platinum | 5 |
| Gold | 4 |
| Tantalum | 4 |

Tissue has a thermal conductivity which is much lower than any of these sensor materials. The gradient layer will have a much lower thermal conductivity than the metal used, perhaps 10% to 1% as much depending on how we make the width and spacing of the grooves and whether we use two sets of grooves perpendicular to one another. For a gradient layer with 10% coverage due to grooves in the material, the thickness of the gradient layer itself might be a maximum of 10% of this, as illustrated in TABLE 3.

TABLE 3

| | Tip Thickness in mils |
|---|---|
| Material | Δ5 = 5° C., P = 20 w |
| Silver | 3 |
| Copper | 3 |
| Aluminum | 2 |
| Magnesium | 1 |
| Silicon | 1 |
| Brass | 1 |
| Iron | 1 |
| Platinum | 1 |
| Gold | 0 |
| Tantalum | 0 |

A few of the most conductive materials would be useful for constructing a reasonable gradient layer heat flow sensor by making grooves in the material. Silver would work well. Copper is harder and less expensive. If made from silver, the top layer would be 30 mils thick, and the bottom 30 mils thick. The bottom would have grooves 10 mils deep milled on one side.

The grooves may also be mechanically or chemically milled with rows of groves or with both rows and columns of grooves. The milled layer can be bonded to the layer with no grooves, leaving air spaces in the grooves. The grooves may also be filled with any material with much poorer thermal conductivity, like plastic or foam if desired. An embodiment of an RF ablation catheter with heat flow sensing capability includes a sensor with a diameter of perhaps 3 mm, and that is fabricated so that one face of the sensor is the tip electrode itself. In other embodiments, the sensor is not the actual tip electrode. The grooves would thus be perhaps 5 mils apart and 10 mils deep.

The surface of the top and bottom layer could be tinned with solder first. One side would be milled, the two sides might be coated with solder flux and then pressed together in the proper orientation and heated to melt the solder and bond the two layers. After the part cools, the remaining flux can be removed by washing it in a suitable solvent. A temperature sensor (e.g. sensors T1, T2) is required in the top and bottom layer. A hole can be drilled vertically through the top layer and down to the middle of the solid part of the bottom layer for its sensor. A hole can be drilled a short distance into the middle of the top layer. A thermocouple or thermistor can then be positioned in place in each hole and bonded with a suitable adhesive such as multicure UV epoxy.

The layers may be made of silicon for a cheaply mass produced sensor. Silicon wafers are usually ½ mm thick, which is about 20 mils. The top may be made of a single large silicon wafer. The bottom is made from a similar wafer which has rows and columns of grooves milled into its face, leaving an array of small very short posts. For example, if we were to make a sensor which is one centimeter square (much larger than typical for a sensor), it might have the characteristics illustrated in TABLE 5.

TABLE 5

| | Tip Thickness in mils | |
|---|---|---|
| Material | Δ5 = 5° C., P = 20 w | |
| Total Thickness | 1 mm | (40 mils) |
| Length | 1 cm | (400 mils) |
| Width | 1 cm | (400 mils) |
| Groove Depth | 0.25 mm | (10 mils) |
| Grove Width | 0.25 mm | (10 mils) |

The silicon wafer can be thinned to reduce the size of the sensor.

The thermal resistivity is R=kA/thickness, where k is the thermal conductivity, A is the area of the face of the sensor, and thickness is the vertical height of the posts of the gradient layer. The thickness of the rest of the two layers can be ignored as, without grooves, its thermal resistance is small in comparison. A single parallel array of grooves provides a thermal resistivity of 5.3 watts/degree C., and two perpendicular arrays of grooves provide a thermal resistivity 0.53 watts/degree C. The sensor could also be made smaller in lateral dimensions with grooves which are narrower and its sensitivity would be greater. When making a silicon sensor requiring chemical milling such as a pressure sensor, the milling is usually done on the back side and the ion implanted resistors or circuitry is placed on the top side. With this heat flow sensor, it is possible to implement the required temperature sensors on each side by ion implantation or by fabricating an IC for a temperature sensor on the top of the top layer and the bottom of the bottom layer, leaving the grooves in the middle of the sandwich. This method of construction would lend itself to manufacture of a very inexpensive sensor.

FIG. 17 illustrates an embodiment of a mapping and ablation system 1723, according to various embodiments of the present subject matter. The illustrated system includes an open-irrigated catheter, but could be used with closed-irrigation catheters or non-irrigation catheters. The illustrated catheter includes an ablation tip 1724 with an RF ablation electrode 1725 and irrigation ports therein. The catheter can be functionally divided into four regions: the operative distal ablation electrode 1725, a main catheter region 1726, a deflectable catheter region 1727, and a proximal catheter handle region where a handle assembly 1728 including a handle is attached. A body of the catheter includes a cooling fluid lumen and may include other tubular element(s) to provide the desired functionality to the catheter. The addition of metal in the form of a braided mesh layer sandwiched in between layers of plastic tubing may be used to increase the rotational stiffness of the catheter.

The deflectable catheter region 1727 allows the catheter to be steered through the vasculature of the patient and allows the probe assembly to be accurately placed adjacent the targeted tissue region. A steering wire (not shown) may be slidably disposed within the catheter body. The handle assembly may include a steering member to push and pull the steering wire. Pulling the steering wire causes the wire to move proximally relative to the catheter body which, in turn, tensions the steering wire, thus pulling and bending the catheter deflectable region into an arc. Pushing the steering wire causes the steering wire to move distally relative to the catheter body which, in turn, relaxes the steering wire, thus allowing the catheter to return toward its form. To assist in the deflection of the catheter, the deflectable catheter region may be made of a lower durometer plastic than the main catheter region.

The illustrated system 1723 includes an RF generator 1729 used to generate the power for the ablation procedure. The RF generator 1729 includes a source 1730 for the RF power and a controller 1731 for controlling the timing and the level of the RF power delivered through the ablation tip 1724. The illustrated system 1723 also includes a fluid reservoir and pump 1732 for pumping cooling fluid, such as a saline, through the catheter and out through the irrigation ports. Some system embodiments incorporate a mapping function. Mapping electrodes may be incorporated into the catheter system. In such systems, a mapping signal processor 1733 is connected to the mapping electrodes to detect electrical activity of the heart. This electrical activity is evaluated to analyze an arrhythmia and to determine where to deliver the ablation energy as a therapy for the arrhythmia. One of ordinary skill in the art will understand that the modules and other circuitry shown and described herein can be implemented using software, hardware, and/or firmware. Various disclosed methods may be implemented as a set of instructions contained on a computer-accessible medium capable of directing a processor to perform the respective method.

FIGS. 18-21 illustrate various processes, such as may be performed in various embodiments of the present subject matter. FIG. 18 illustrates a method for determining thermal properties of tissue, according to various embodiments. Such a method may be automatically performed by the controller 1731 for example, may be performed by a user using an ablation system, or may be performed as a combination of automatic and manual steps. At 1834, the temperatures T₁ and T₂ are measured to obtain a temperature gradient from a more distal region to a more proximal region. For example, because the RF electrode tip has a high thermal conductivity, the T₁ near the tip closely represents the temperature of the tissue at the electrode-tissue interface. Temperature T₂ is measured in a direction of expected heat flow. At 1835, the heat flow from a point corresponding to T₁ to a point corresponding to T₂ is calculated, to determine the heat flow (P_{CONV}) that is attributed to convective cooling to blood or irrigation fluid. At 1836, the value of P_{CONV} and the known generated RF energy P_{E} is used to calculate the power dissipated into the tissue (P_{T}=P_{E}−P_{CONV}). This provides an accurate estimation of the RF power dissipated into the tissue, which provides the ability to accurately estimate tissue lesions formed by the RF power.

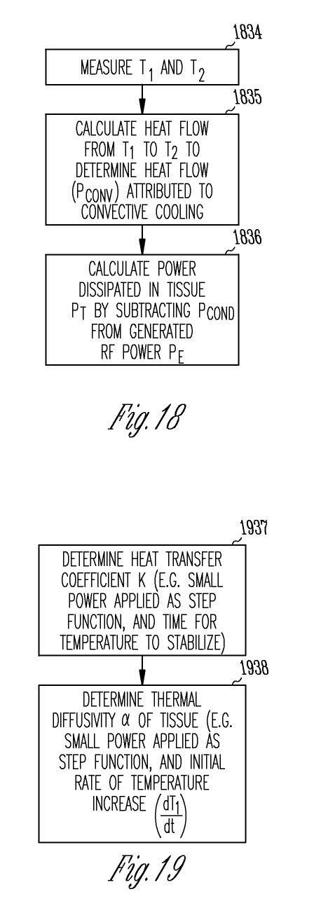

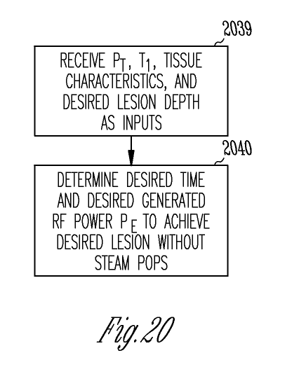

FIG. 19 illustrates a method for determining thermal properties of tissue, according to various embodiments. Such a method may be automatically performed by the controller 1731 for example, may be performed by a user using an ablation system, or may be performed as a combination of automatic and manual steps. At 1937, the heat transfer coefficient κ is determined. In some embodiments, a small amount of RF power is applied as a step function. The time required for the temperature of the tissue, as measured by the most distal thermocouple T₁, is determined. At 1938, the thermal diffusivity a of the tissue is determined. In some embodiments, a small amount of RF power is applied as a step function, and the initial rate of temperature increase of the tissue is determined, using measurements by the most distal thermocouple T₁.

FIG. 20 illustrates a method for determining the time and power for RF ablation, according to various embodiments. At 2039, the RF power dissipated into the tissue (P_{T}), the most distal thermocouple temperature (T₁), tissue thermal characteristics, and the desired lesion size for a desired ablation procedure are inputted or otherwise received. The desired time and amplitude profile for the generated RF power (P_{E}) is determined using these inputs to achieve the desired lesion size without steam pops. Such a method may be automatically performed by the controller 1731 for example, may be performed by a user using an ablation system, or may be performed as a combination of automatic and manual steps.

This application is intended to cover adaptations or variations of the present subject matter. It is to be understood that the above description is intended to be illustrative, and not restrictive. The scope of the present subject matter should be determined with reference to the appended claims, along with the full scope of legal equivalents to which such claims are entitled.

What is claimed is:

1. A system for ablating tissue, comprising:
an electrode configured for use to deliver RF power to ablate the tissue; and
a heat flow sensor configured to provide a measurement of heat flow from the electrode to blood or irrigation fluid.

2. The system of claim 1, further comprising a heat sink and a gradient layer positioned between the heat sink and the electrode, wherein the heat flow sensor includes a first temperature sensor positioned on the electrode and a second temperature sensor positioned on the heat sink.

3. The system of claim 2, wherein the heat sink is configured to be cooled by blood when positioned to perform an ablation procedure.

4. The system of claim 2, wherein the heat sink is configured to be cooled by cooling fluid in a closed-irrigation ablation system.

5. The system of claim 2, wherein the heat sink is configured to be cooled by cooling fluid in an open-irrigation ablation system.

6. The system of claim 1, further comprising at least a second electrode configured for use to deliver RF power, and a second heat flow sensor configured to measure heat flow from an interface between the second electrode to blood or irrigation fluid.

7. The system of claim 6, further comprising at least a third electrode configured for use to deliver RF power, and a third heat flow sensor configured to measure heat flow from an interface between the second electrode to blood or irrigation fluid.

8. The system of claim 1, wherein the heat flow sensor includes:
a first temperature sensor positioned distally on the electrode near tissue to be ablated when the electrode is positioned to perform an ablation procedure; and
a second temperature sensor positioned on the electrode proximally with respect to the first temperature sensor for use to sense heat flow from the first temperature sensor to the second temperature sensor.

9. The system of claim 8, wherein the system is a non-irrigation ablation system, a closed-irrigation system, or an open-irrigation system.

10. The system of claim 8, wherein:
the electrode is formed using a material;
the electrode includes a distal portion formed using the material and a proximal portion formed using the material;
the first temperature sensor is positioned on the proximal portion, and the second temperature sensor is positioned on the distal portion;
the distal portion has a proximal face;
the proximal portion has a distal face in contact with the proximal face of the distal portion; and
at least one of the distal face or the proximal face has a pattern of grooves formed therein to reduce thermal conductivity between the first temperature sensor and the second temperature sensor.

11. The system of claim 1, further comprising a heat sink with a distal face, wherein:
the electrode has a proximal face;
the heat flow sensor is configured to sense heat flow between the electrode and the heat sink; and
the heat flow sensor includes a first block of a first thermoelectric heat pump material and a second block of second thermoelectric heat pump material positioned between the heat sink and the electrode, wherein:
the proximal face of the electrode is in contact with both blocks;
the distal face of the heat sink is in contact with both blocks;
a first electrical conductor is connected to the first block near an interface with the distal face of the heat sink and a second electrical conductor is connected to the second block near the interface;
a voltage difference between the first and second electrical conductors provides an indication of a temperature difference between the interface of the first block and the distal face and the interface of the second block and the distal face; and
the temperature difference provides an indication of heat flow.

12. The system of claim 1, further comprising:
an RF source configured to generate RF power connected to the electrode (P_{E}) to ablate tissue;
a controller configured to control a level of RF power and a duration for an ablation procedure, wherein the controller is programmed to implement a process to estimate RF power dissipated in tissue (P_{T}), wherein the process includes:
calculating convective heat flow (P_{CONV}) from the tissue through the electrode to the blood or the irrigation fluid to cool the electrode; and
calculating the RF power dissipated in tissue (P_{T}) by subtracting P_{CONV} from P_{E}.

13. The system of claim 12, wherein the controller is programmed to implement a process to estimate thermal properties of tissue, wherein the thermal properties include at least one of a heat transfer coefficient or thermal diffusivity.

14. The system of claim 12, wherein the controller is programmed to implement a process to determine the duration and the RF power (P_{E}) to achieve a desired lesion without steam pops.

15. A method, comprising:
measuring convective heat flow (P_{CONV}) from the tissue through the electrode to the blood or the irrigation fluid to cool the electrode; and
calculating RF power dissipated in tissue (P_{T}) by subtracting P_{CONV} from generated RF power (P_{E}) for an ablation procedure.

16. The method of claim 15, wherein measuring P_{CONV} includes:
measuring a first temperature near an interface between the electrode and the tissue when the electrode is in position to ablate the tissue; and
measuring a second temperature in a direction of expected convective heat flow; and using the first and second temperatures to calculate P_{CONV}.

17. The method of claim 16, wherein measuring convective heat flow (P_{CONV}) from the tissue through the electrode includes measuring P_{CONV} through a gradient layer between the electrode and a heat sink, wherein the heat sink is in contact with the blood or the irrigation fluid, and the first and second temperatures are measured on opposite sides of the gradient layer.

18. The method of claim 16, further comprising controlling a duration and a level of P_{E} for performing the ablation procedure using the calculated P_{T}.

19. The method of claim 18, further comprising:
estimating thermal properties of tissue, wherein the thermal properties include at least one of a heat transfer coefficient or thermal diffusivity; and
using the estimated thermal properties of tissue with the calculated P_{T} to control the duration and the level of P_{E} for performing the ablation procedure.

20. The method of claim 19, wherein:
estimating thermal properties includes using the first temperature to estimate the thermal properties of the tissue;
calculating P_{T} includes automatically calculating P_{T} by subtracting P_{CONV} from generated RF power (P_{E}) for the ablation procedure; and
automatically determining the duration and the level of P_{E} for performing the ablation procedure using the calculated P_{T}.
